(19) United States
(12) Patent Application Publication
(10) Pub. No.: US 2008/0063617 A1
(43) Pub. Date: Mar. 13, 2008

(54) COSMETICS FORMULATIONS

(76) Inventors: John M. Abrahams, Scarsdale, NY (US); Weiliam Chen, Mount Sinai, NY (US)

Correspondence Address: SCHWEGMAN, LUNDBERG & WOESSNER, P.A., P.O. BOX 2938, MINNEAPOLIS, MN, 55402, US

(21) Appl. No.: 11/470,818

(22) Filed: Sep. 7, 2006

(51) Int. Cl. A61K 8/73

(52) U.S. Cl. 424/70.13

(57) ABSTRACT

A cosmetic formulation containing an alkylated chitosan derivative, a polybasic carboxylic acid or an oxidized polysaccharide, and water, for moisturizing and/or softening skin is provided. The formulation is in the physical state of a viscous liquid or a gel, and when applied to skin, serves to moisturize and soften the skin. One embodiment of the formulation comprises PEG-chitosan and hyaluronic acid at a pH of about 3.5 to about 5.5. The formulation may be applied to skin as a bulk composition, on a towel or wipe, or in the form of a facial foam mask.

### FIELD OF THE INVENTION

The field of the invention is cosmetic formulations comprising alkylated chitosans and polyfunctional carboxylic acids or aldehydes in water that form viscous liquids or gels for use as skin moisturizers and softeners.

### BACKGROUND OF THE INVENTION

Chitosan is a semi-synthetic polysaccharide derivative formed by deacetylation of chitin, a major component of arthropod skeletons and fungal cell walls. An analog of cellulose, chitosan is formed of β-1,4-linked glucose molecules in which the 2-hydroxyl group of each glucose unit is replaced by an amino group. Thus, chitosan is a cationic polysaccharide in acidic aqueous solution, the amino groups being protonated at acidic pHs to form positive charges. Chitosan, without further derivatization, is largely insoluble at neutral pH. However, functional derivatization of chitosan can increase water solubility of the polymer at neutral pH and alter other properties of the polymer as well.

The properties of chitosan can be modified through chemical derivatization. One such type of derivatization includes bonding chitosan to chemical moieties that increase water-solubility, such as through introduction of hydrophilic groups, either ionic or non-ionic. An example of an ionic hydrophilic derivative of chitosan is acrylated chitosan, wherein the introduced carboxyl groups, being ionizable at neutral or alkaline pHs, convert the cationic chitosan to an amphipathic, water-soluble derivative. An example of a non-ionic hydrophilic derivative of chitosan is a poly(oxyethylene)chitosan, also known as PEG-chitosan, wherein the multiple oxygen atoms of the poly(oxyethylene) chain do not bear a formal electrical charge but can form hydrogen bonds with water molecules. One useful property of hydrophilic chitosan derivatives is the high viscosity of aqueous (water) solutions formed by relatively low concentrations of the chitosan derivatives.

Acrylated chitosans are formed by the reaction of acrylic acid or its salts with chitosan. It is generally believed that the major mode of reaction of the acrylic acid or salt is via Michael addition of the chitosan amino groups to the β-carbon of the α,β-unsaturated carboxylate. This reaction does not destroy the cationic character of the amino groups, as they continue to exist in amino form, not as amides. The addition of the acrylate carboxylic acid groups to the polymer molecule thus serves to convert chitosan into an amphipathic polymer, bearing both positive and negative electrical charges, that tends to increase water solubility. For example, refer to H. Sashiwi, et al., Biomacromolecules (2003 September-October), 4(5), 1250-4.

Poly(oxyalkylene)chitosans such as poly(oxyethylene)chitosans, also known as poly-ethyleneglycol-chitosans or PEG-chitosans or PEG-grafted chitosans, are formed by coupling of activated forms of polyethylene glycol with chitosan. The polyethylene glycols themselves are formed by the polymerization of alkylene oxides (epoxides) such as ethylene oxide. Polyethyleneglycols may be obtained in a wide variety of molecular weights, with various structural features such as activated end groups, hydrolysable linkages, and others. For example, refer to the Nektar PEG catalog that lists a wide variety of the Shearwater functionalized PEGs, at www.nektar.com/pdf/nektar_catalog.pdf (as of Aug. 24, 2006). The covalent addition of these hydrophilic but non-ionic poly(oxyalkylene) or poly(ethyleneglycol) groups to the chitosan molecule alters its physical properties. U.S. Pat. No. 6,602,952 describes the preparation of poly(alkyleneoxide)chitosan derivatives and their use in the formation of hydrogels. U.S. Pat. No. 6,730,735 and U.S. Published Application No. 2004/0166158 describe chitosan derivatives comprising a poly(oxyethylene)chitosan wherein the poly(oxyethylene) or poly(ethyleneglycol) unit is coupled to the chitosan via an acrylic acid functionality. U.S. Published Application No. 2004/0156904 describes the use of a poly(ethyleneglycol)chitosan with a water insoluble polymer in a non-aqueous solvent.

Other methods have been described for the preparation of chitosan derivatives and their incorporation into various formulations. Published PCT application WO2005/113608 and published U.S. Patent Application No. 2005/0271729 discuss the crosslinking of chitosan and hyaluronan (also known as hyaluronic acid) at a pH of at least 7.2 in the presence of a carbodiimide coupling reagent to form a gel. Hyaluronan is an acidic linear polysaccharide formed of β-1,3 linked dimeric units, the dimeric units consisting of an 2-acetamido-2-deoxyglucose and D-gluconic acid linked in a β-1,4 configuration.

Certain chitosan derivatives have been used in cosmetic applications. For example, U.S. Pat. No. 7,053,068 describes reaction products of chitosan and 2-iminothiolane and their use as ingredients of cosmetics. U.S. Pat. No. 6,458,938 describes the use of chitosan to which poly(ethyleneglycol) units have been added as an ingredient of cosmetics. U.S. Pat. No. 4,822,598 describes chitosan bearing substituents with quaternary ammonium groups as components of cosmetics. U.S. Pat. No. 3,953,608 describes the use of the reaction product of chitosan with organic diacid anhydrides as ingredients of cosmetics, as does U.S. Pat. No. 3,879,376. U.S. Pat. No. 4,528,283 discusses glyceryl chitosan derivatives formed by reaction of chitosan with glycidol and their use in cosmetics.

Certain formulations comprising alkylated chitosan derivatives and polybasic carboxylic acids or oxidized polysaccharides that form gels are disclosed in co-pending application Ser. No. 11/425,280, filed Jun. 20, 2006, by the inventors herein.

Cosmetic components that act to moisturize or soften skin are well known. For example, refer to “Skin Care and Moisturizers,” The World of Skin Care, an online reference by Dr. John Gray, provided by the P&G Skincare Research Center, at http://www.pg.com/science/skincare/Skin_{—tws}_{—}102.htm;jsessionid=N1PGE1VIIK135QFIAJ1S0HWAVABHMLKG (as of Aug. 24, 2006); or refer to “Moisturizer” in Wikipedia at http://en.wikipedia.org/wiki/Moisturizer. Absorption of water by living cells tends to increase their volume, and absorption of water by skin proteins softens their texture and makes them less horny and brittle. These effects, among others, serve to produce the effects of smoothness and softness of the skin.

### SUMMARY

It has surprisingly been found by the inventors herein that a formulation comprising a substantially water-soluble alkylated chitosan derivative such as an acrylated chitosan or a poly(oxyalkylene)chitosan, the formulation also comprising a polybasic carboxylic acid or an oxidized polysaccharide, in water solution, is useful as a cosmetic for moisturizing or softening living human skin when applied to the skin. The present invention provides a cosmetic formulation comprising a viscous solution or a gel that is formed by the interaction of the alkylated chitosan derivative and the polybasic carboxylic acid or oxidized polysaccharide in water. The viscosity of the formulation is greater than the viscosity of either of its components alone at comparable concentrations and temperatures, and serves to hold water in close contact with skin to which the formulation is applied. The polymeric components of the formulation may themselves also exert an effect upon skin.

In one aspect, a formulation of the present invention comprises a poly(oxyalkylene)chitosan. An example is poly(oxyethylene)chitosan or, synonymously, PEG-chitosan. In another aspect, a formulation comprises an acrylated chitosan, a chitosan derivative formed by reaction of chitosan with acrylic acid or its salts.

A formulation of the invention also comprises a polybasic carboxylic acid or an oxidized polysaccharide. In one aspect of the invention, the formulation comprises a polybasic carboxylic acid. A polybasic carboxylic acid can be an acidic polysaccharide (which may be naturally occurring or synthetic); that is, a polymer comprising carbohydrate monomeric units at least some of which incorporate carboxylic acid groups. An example is hyaluronic acid; another example is carboxymethylcellulose. In another aspect the polybasic carboxylic acid can be a low molecular weight di-, tri- or tetra-carboxylic acid; examples are (C₃-C₁₂)-alkane-α,ω-dicarboxylic acids such as adipic acid, tri-carboxylic acids such as citric acid, and tetra-carboxylic acids such as ethylenediamine-tetraacetic acid.

In another aspect of the invention, the formulation comprises an oxidized polysaccharide. An oxidized polysaccharide contains carboxaldehyde groups, such as are formed by periodate oxidation of a compound with a vicinal diol unit, such as poly-glycose. Examples include oxidized starch, oxidized dextran, or oxidized hyaluronic acid.

These formulations are useful for moisturizing, softening and smoothing human skin when applied thereto.

The invention provides a method of softening and moisturizing skin, comprising holding a formulation of the invention in close contact with skin for a period of time, typically about 10 seconds to about 60 minutes.

The invention further provides a method for preparing a formulation of the invention, wherein a solution of the chitosan derivative and a solution of the polybasic carboxylic acid or of the oxidized polysaccharide are mixed, either directly on the skin, or prior to application to the skin. The mixing may be carried out in advance and the formulation kept in moist form until applied to the skin. Alternatively, the components may be applied separately to the skin and mixed in situ, the partial gelation or marked viscosity increase occurring in the time course of a few minutes, typically less than 12 minutes.

The invention further provides a use of cosmetic composition comprising a formulation of the invention for moisturizing or softening skin. Also, use of a disposable towel or skin wipe, or a foam facial mask, comprising the formulation of claim 1 for moisturizing or softening skin is provided. Use of the formulation further comprising a medicament useful for treatment of a skin malcondition is also provided.

### BRIEF DESCRIPTION OF THE DRAWINGS

FIG. 1 is a graph showing the viscosity of various concentration of hyaluronan (hyaluronic acid) as a function of shear rate in comparison with the viscosity of a formulation of the invention comprising PEG-chitosan plus hyaluronan at a pH of 3.5 to 5.5.

FIGS. 2, 3, and 4 are bar graphs showing the relative viscosities of a range of hyaluronan solutions versus a formulation of the invention comprising PEG-chitosan plus hyaluronan at three different shear rates.

FIG. 5 is a graphical depiction of the results of a cell viability assay in the presence of a formulation of the invention comprising PEG-chitosan plus hyaluronan compared to control.

FIG. 6 is a photograph of three solutions showing their qualitative visual viscosities.

### DETAILED DESCRIPTION OF THE INVENTION

### Definitions

“Chitosan,” as the term is used herein, refers to deacetylated chitin, the natural product found in fungi and crustacean shells. Chitosan is polymeric D-glucosamine (2-amino-2-deoxyglucose) linked in the β-1,4 configuration.

An example of a section of a chitosan chain has the following chemical structure, wherein the number of glucosamine units may range from only a few upwards into the hundreds:

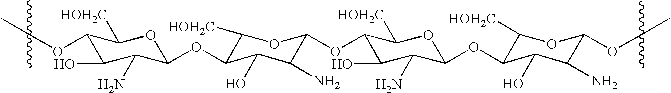

Chitosan is commercially available in a wide range of purities, degrees of polymerization, and degrees of deacetylation, from a number of suppliers. It is biocompatible and biodegradable, and has been used to form films, in biomedical devices and to form microcapsule implants for controlled release in drug delivery. See, e.g., S. Hirano et al., Biochem. Sys. Ecol., 19, 379 (1991); A. D. Sezer, Microencapsulation, 16, 687 (1999); A. Bartkowiak et al., Chem. Mater. 11., 2486 (1999); T. Suzuki et al., Biosci. Bioeng., 88, 194 (1999).

When referring to the “molecular weight” of a polymeric species such as an alkylated chitosan, a weight-average molecular weight is being referred to herein, as is well known in the art.

A “degree of substitution” of a polymeric species refers to the ratio of the average number of substituent groups, for example an alkyl substituent, per monomeric unit of the polymer as defined.

A “degree of polymerization” of a polymeric species refers to the number of monomeric units in a given polymer molecule, or the average of such numbers for a set of polymer molecules.

As the term is used herein, an “alkylated chitosan” is a molecular entity formed by reaction of chitosan with carbon-containing molecules. For example, methylation of chitosan, in which bonds are formed between methyl radicals or groups and atoms within the chitosan molecule, such as nitrogen, oxygen or carbon atoms, provides an alkylated chitosan within the definition used herein. Other carbon-containing groups may likewise be chemically bonded to chitosan molecules to produce an alkylated chitosan. For example, poly(oxyalkylene)chitosan and acrylated chitosan are alkylated chitosans within the meaning of the term herein.

A “poly(oxyalkylene)chitosan” is a variety of alkylated chitosan as defined herein. A “poly(oxyalkylene)” group is a polymeric chain of atoms wherein two carbon atoms, an ethylene group, are bonded at either end to oxygen atoms. The carbon atoms of the ethylene group may themselves bear additional radicals. For example, if each ethylene group bears a single methyl group, the resulting poly(oxyalkylene) group is a poly(oxypropylene) group. If the ethylene groups are unsubstituted, the poly(oxyalkylene) group is a poly(oxyethylene) group. A poly(oxyethylene) group may be of a wide range of lengths, or degrees of polymerization, but is of the general molecular formula of the structure [—CH₂—CH₂—O—CH₂—CH₂—O—]ₙ, where n may range from about 3 upwards to 10,000 or more. Commonly referred to as “polyethyleneglycol” or “PEG” derivatives, these polymeric chains are of a hydrophilic, or water-soluble, nature. Thus, a poly(oxyalkylene)chitosan is a chitosan derivative to which poly(oxyalkylene) groups are covalently attached. A terminal carbon atom of the poly(oxyalkylene) group forms a covalent bond with an atom of the chitosan chain, likely a nitrogen atom, although bonds to oxygen or even carbon atoms of the chitosan chain may exist. Poly(oxyethylene)chitosan is often referred to as “polyethyleneglycol-grafted chitosan” or “PEG-g-chitosan.”

The end of the poly(oxyethylene) chain that is not bonded to the chitosan backbone may be a free hydroxyl group, or may comprise a capping group such as methyl. Thus, “polyethylene glycol” or “poly(oxyethylene)” or “poly(oxyalkylene)” as used herein includes polymers of this class wherein one, but not both, of the terminal hydroxyl groups is capped, such as with a methyl group. In a specific method of preparation of the poly(oxyethylene)chitosan, use of a polyethyleneglycol capped at one end, such as MPEG (methyl polyethyleneglycol) may be advantageous in that if the PEG is first oxidized to provide a terminal aldehyde group, which is then used to alkylate the chitosan nitrogen atoms via a reductive amination method, blocking of one end of the PEG assures that no difunctional PEG that may crosslink two independent chitosan chains is present in the alkylation reaction. It is preferred to avoid crosslinking in preparation of the poly(oxyethylene)chitosan of the present invention. A representative structure of a poly(oxyethylene)chitosan is shown below.

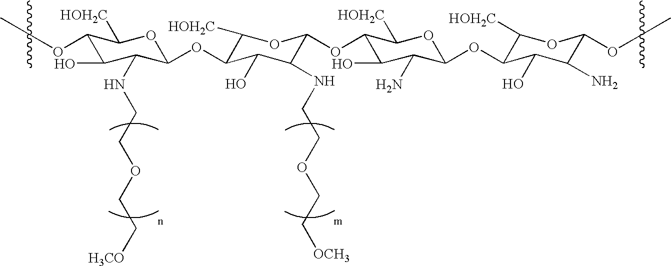

An “acrylated chitosan” as the term is used herein is an alkylated chitosan wherein acrylates have been allowed to react with, and form chemical bonds to, the chitosan molecule. An acrylate is a molecule containing an α,β-unsaturated carbonyl group; thus, acrylic acid is prop-2-enoic acid. An acrylated chitosan is a chitosan wherein a reaction with acrylates has taken place. The acrylate may bond to the chitosan through a Michael addition of the chitosan nitrogen atoms with the acrylate. An example of the chemical structure of a segment of an acrylated chitosan polymer is shown below.

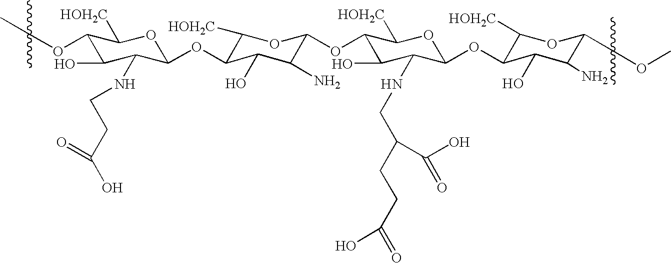

As used herein, a “polybasic carboxylic acid” means a carboxylic acid with more than one ionizable carboxylate residue per molecule. The carboxylic acid may be in an ionized or salt form within the meaning of the term herein. A polybasic carboxylic acid includes a dibasic, tribasic, or tetrabasic low molecular weight carboxylic acid within the meaning herein. By “low molecular weight” is meant that the compound has a molecular weight less than about 1000, that is, it is not a polymeric material. An alkane-α,ω-dicarboxylic acid is an example of a class of polybasic carboxylic acid, and adipic acid is a more specific example. Disodium adipate is another example. A tribasic carboxylic acid is another example of a class of polybasic carboxylic acid, and citric acid is a more specific example. A tetrabasic carboxylic acid is another example of a class of polybasic carboxylic acid, and ethylenediamine-tetraacetic acid is a more specific example.

Alternatively, the polybasic carboxylic acid may have hundreds or thousands of ionizable carboxylate groups per molecule as when the molecule is polymeric in character; for example, hyaluronan, also known as hyaluronic acid, is also a polybasic carboxylic acid within the meaning assigned herein. The hyaluronan or hyaluronic acid may be in an ionized or salt form, for example sodium hyaluronate, which is a polybasic carboxylic acid within the meaning of the term as used herein.

As used herein, the term “acidic polysaccharide” refers to a polymeric carbohydrate comprising carboxylic acid groups. The polymeric carbohydrate can be naturally occurring, or can be synthetic or semi-synthetic. Examples of acidic polysaccharides are hyaluronan and carboxymethyl cellulose. An oxidized hyaluronan, that is, hyaluronan that has been treated with an oxidizing agent, such as sodium periodate, that cleaves vicinal diol moieties and provides aldehyde groups so is an oxidized polysaccharide is also an acidic polysaccharide within the meaning herein.

As used herein, the term “oxidized polysaccharide” refers to a polymeric carbohydrate that has undergone treatment with an oxidizing reagent, such as sodium periodate, that cleaves vicinal diol moieties of the carbohydrate to yield aldehyde groups. An oxidized hyaluronan, that is, hyaluronan that has been treated with an oxidizing agent, such as sodium periodate, that cleaves vicinal diol moieties and provides aldehyde groups, is an example of an oxidized polysaccharide within the meaning herein. An oxidized dextran, that is, dextran that has been treated with an oxidizing agent, such as sodium periodate, that cleaves vicinal diol moieties and provides aldehyde groups, is another example of an oxidized polysaccharide within the meaning herein.

### Description

In an embodiment of a use of a formulation according to the present invention, a solution of an alkylated chitosan derivative and a polybasic carboxylic acid or an oxidized polysaccharide in water forms a viscous solution or partial gel that, when applied to living skin tissue, serves to moisturize and soften the skin. By a “viscous solution or a partial gel” is meant a material that either flows very slowly under the influence of gravity, or does not flow noticeably unless it is subjected to shaking or other impetus. The viscous solution or partial gel may be thixotropic, that is, it undergoes a reduction in its normally high viscosity when shaken, stirred or otherwise mechanically disturbed, but readily recovers its original condition on standing. Typically, a concentration of only a few percent by weight of the chitosan derivative and of less than a percent by weight of hyaluronan is required to increase the viscosity of water to this highly viscous or gelled state.

The highly viscous liquid or gel, when applied to skin, is very effective in holding water in close contact with the skin for extended periods of time, as a substantial volume of water is retained within the formulation of the invention. Furthermore, it is believed that the chitosan derivative itself, and possibly the polyfunctional carboxylic acid or aldehyde, may play a role in moisturizing and softening the skin beyond its passive role in holding water in a gelled state against the skin.

It should be noted that these polymeric components are generally regarded as safe for application to skin.

As described in the Examples, human subjects who applied formulations to their skin, for example to their forearms, and allowed the formulation to remain in contact with the skin for a period of minutes prior to removing it, noted that the area of skin to which the formulation had been applied was perceived to be remarkably smooth and soft for some time after removal of the formulation.

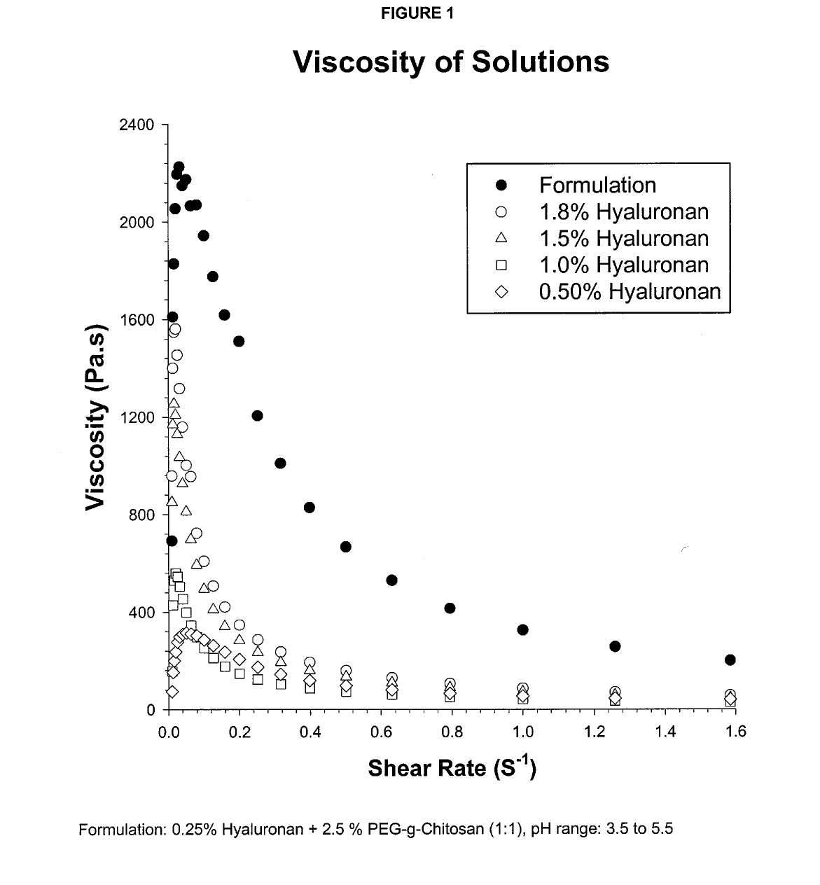

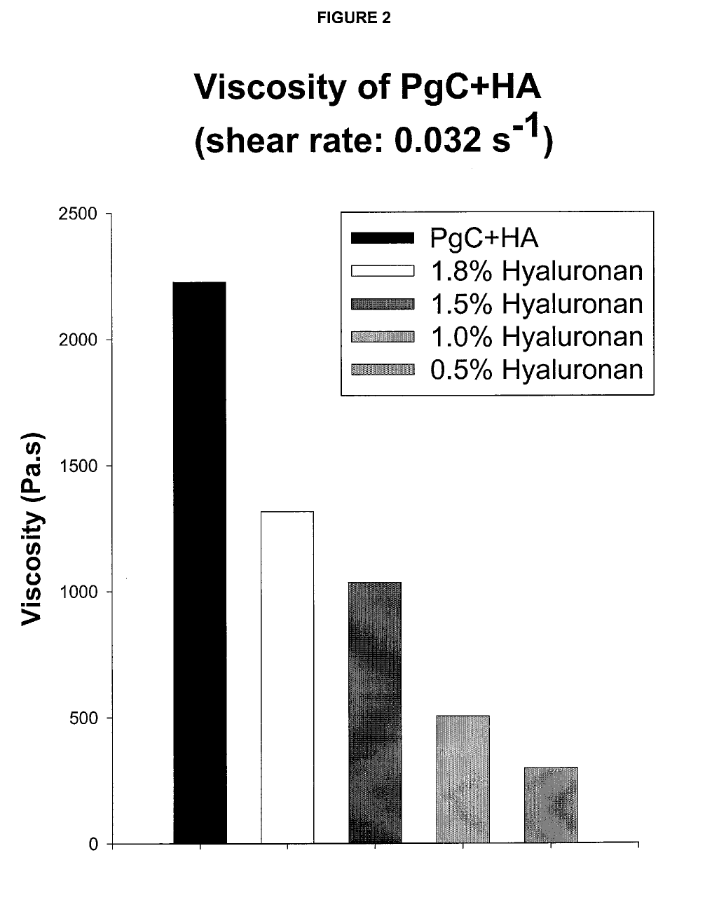

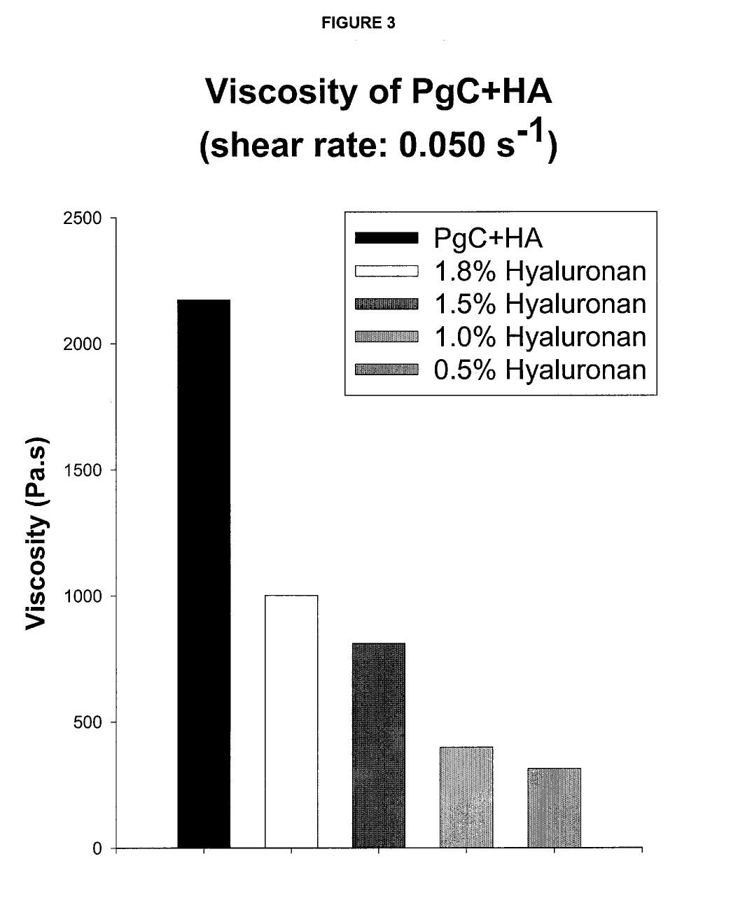

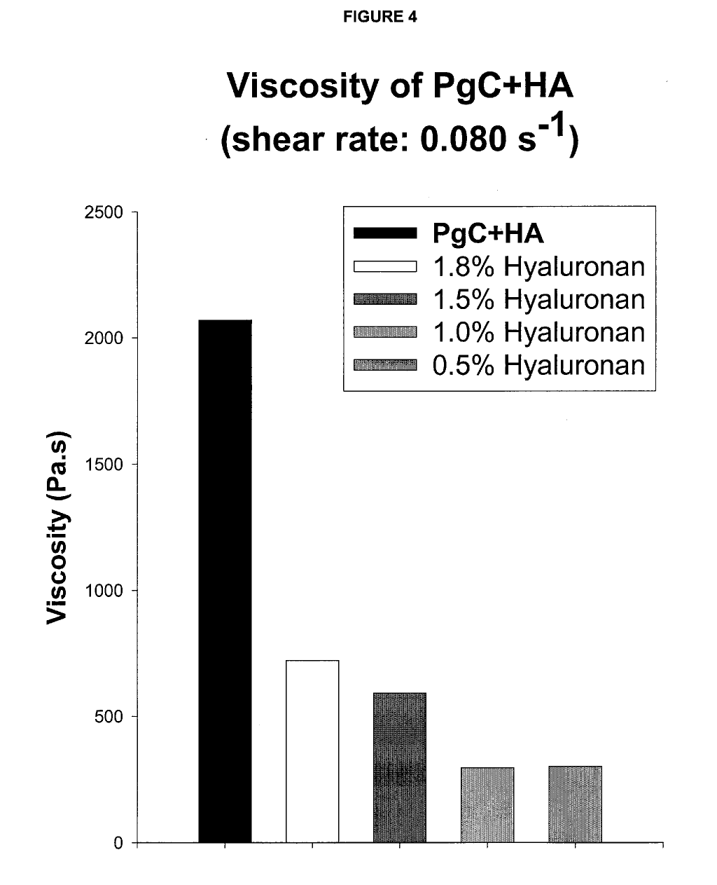

One embodiment of a formulation that can be used according to the method of the invention comprises PEG-chitosan and hyaluronan (hyaluronic acid) in water at a pH of about 3.5 to about 5.5, and comprises at most a few percent by weight of both the PEG-chitosan and of the hyaluronan. Even lower concentrations of the polymeric materials in the water are effective to produce a gel or viscous liquid suitable for moisturizing and/or softening skin. For example, water containing about 2.5 wt % PEG-chitosan and about 0.25 wt % hyaluronan, at a pH of about 3.5 to about 5.5, effectively forms a highly viscous liquid or a gel suitable for the use of the invention. Referring to FIG. 1, a graph depicts the viscosity of this composition relative to concentrations of hyaluronan alone ranging from 0.5 wt % to 1.8 wt %. At shear rates ranging from less than 0.1 sec⁻¹ to 1.6 sec⁻¹, the formulation of the invention has a higher viscosity than any of the pure hyaluronan compositions. FIGS. 2, 3, and 4 depict these data for individual shear rates in bar graph form, again indicating the increase in viscosity resulting from mixing the two polymers. It should be noted that the viscosity of 2.5 wt % PEG-chitosan in water without any hyaluronan is lower than the viscosity of 0.1% hyaluronan (˜0.3 Pa·s at a shear rate of 0.6 sec−1).

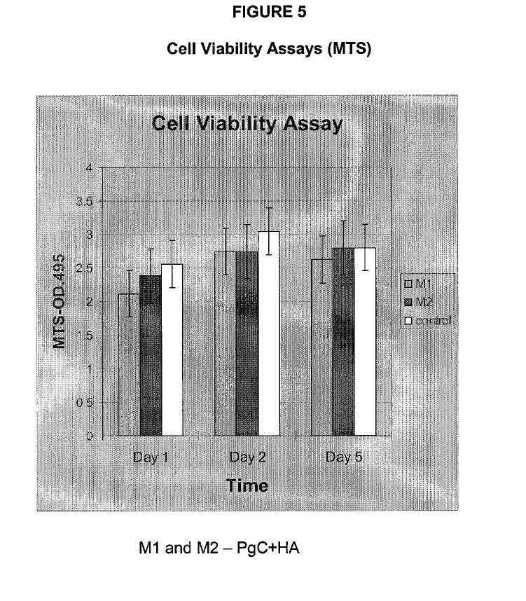

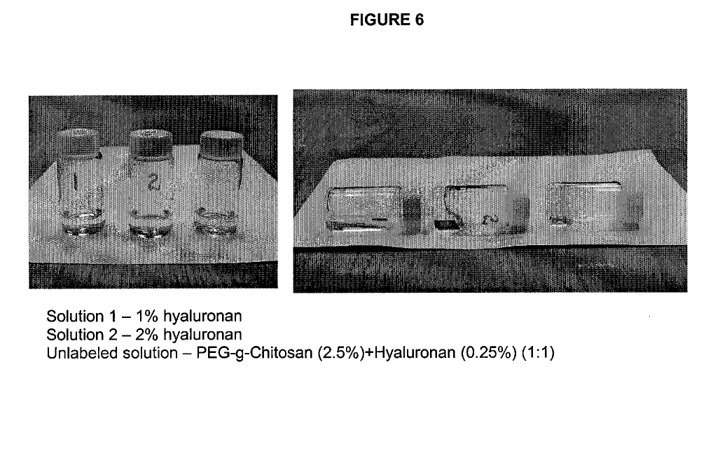

FIG. 5 provides the results of a cell viability assay showing that samples of the PEG-chitosan plus hyaluronan formulation (M1, M2) do not diminish the viability of living cells in comparison to control. This supports the recognition that these materials are generally regarded as safe for skin application.

A similar formulation was obtained using the PEG-chitosan and carboxymethylcellulose, oxidized dextran, or oxidized hyaluronan in water. A stable semi-gel or highly viscous liquid resulted from mixing the two components, the gel forming or the solution markedly increasing in viscosity within a few minutes after mixing. Concentrations are similar to those used in the PEG-chitosan/hyaluronan formulation; PEG-chitosan concentration can be in the 1-10% range, preferably in the 2-5% range, with carboxymethylcellulose, oxidized dextran, and oxidized hyaluronan in the 0.1-5% range, preferably in the 0.5-2.5% range. The pH of the formulation resulting from mixing of the components is preferably in the 3.5 to 5.5 range. If necessary, the pH can be adjusted from a higher pH into the 3.5-5.5 range using a dilute mineral acid, for example about 0.1M hydrochloric acid.

Another embodiment of a formulation according to the invention comprises an acrylated chitosan and a low molecular weight polybasic carboxylic acid. Examples of such carboxylic acids are adipic acid, citric acid, and ethylenediamine-tetraacetic acid. Similarly to the formulation comprising the hyaluronic acid, these formulations rapidly increase in viscosity subsequent to mixing, the final product being either a flowable but highly viscous liquid, or a semi-gel, depending on the concentrations of the reagents in the water solution. Specific concentrations of the components in water are, for the acrylated chitosan, about 1% to about 10%, and for the low molecular weight polybasic carboxylic acid, about 0.1% to about 5%. Again, the preferred pH is in the 3.5 to 5.5 range, and the solution may be adjusted downward in pH to this range using a dilute mineral acid, for example, 0.1M hydrochloric acid.

A cosmetic preparation according to the present invention comprises a formulation of the present invention. For example, a cosmetic preparation may include the highly viscous liquid or gel of the present invention in addition to other cosmetics ingredients, including but not limited to dyes, fragrances, emollients, tanning agents, vitamins or other nutrients, phytochemicals or other natural products, thickeners, dispersants, proteins, peptides, solvents, anti-oxidants, or the like. The preparation may be packaged in bulk, or alternatively, may be placed on a disposable towel or skin wipe kept in a moist state, for example, by packaging in a water-impermeable packing material such as plastic or foil.

A facial foam mask according to the present invention comprises a formulation of the present invention. A facial foam mask is a product made from an aqueous foam stabilized by polymeric ingredients that is frozen and lyophilized to dryness. Upon rewetting, the lyophilized foam is applied to the face to provide moisturizing effects. For example, refer to http://www.butymate.com/ wherein a mask of this type is described. In the past, collagen has often been used for such masks. However, with the present concern about BSE and prions, collagen may no longer be favored or acceptable for such uses. A facial foam mask according to the present invention comprises a lyophilized formulation of the invention that solidifies into a dry foam during the lyophilization process. Upon rewetting, it is applied to facial skin and held there for a period of time to produce a softening or moisturizing effect.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present invention, thus generally described, will be understood more readily by reference to the following examples, which are provided by way of illustration and are not intended to be limiting of the present invention.

### EXAMPLES

### Example 1

### Preparation of Oxidized Dextran

Dextran (5 g) was dissolved in 400 mL of distilled H₂O, then 3.28 g of NaIO₄ dissolved in 100 mL ddH₂O was added. The mixture was stirred at 25° C. for 24 hrs. 10 ml of ethylene glycol was added to neutralize the unreacted periodate following by stirring at room temperature for an additional hour. The final product was dialyzed exhaustively for 3 days against doubly distilled H₂O, then lyophilized to obtain a sample of pure oxidized dextran.

### Example 2

### Analyses of Oxidized Dextran

The degree of oxidation of the oxidized dextran was determined by quantifying the aldehyde groups formed using t-butyl carbazate titration via carbazone formation. A solution of oxidized dextran (10 mg/ml in pH 5.2 acetate buffer) was prepared; and a 5-fold excess tert-butyl carbazate in the same buffer was added and allowed to react for 24 hrs at ambient temperature, then a 5-fold excess of NaBH₃CN was added. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 7.9 ppm (proton attached to tert-butyl) and 4.9 ppm (anomeric proton of dextran).

### Example 3

Gelation of an Oxidized Dextran/Acrylated Chitosan Hydrogel

A 1 mL sample of 2% aqueous oxidized dextran in water solution was mixed with 1 mL of a 2% aqueous acrylated chitosan solution. The mixture was gently stirred for 10 seconds. Gelation occurred within 30 seconds at ambient temperature.

### Example 4

Preparation of Oxidized Hyaluronan

Sodium hyaluronan (1.0 gram) was dissolved in 80 ml of water in a flask shaded by aluminum foil, and sodium periodate (various amounts) dissolved in 20 ml water was added dropwise to obtain oxidized hyaluronan (oHA) with different oxidation degrees. The reaction mixture was incubated at ambient temperature and 10 ml of ethylene glycol was added to neutralize the unreacted periodate following by stirring at room temperature for an additional hour. The solution containing the oxidized hyaluronan was dialyzed exhaustively for 3 days against water, then lyophilized to obtain pure product (yield: 50-67%).

### Example 5

### Analyses of Oxidized Hyaluronan

The degree of oxidation of oxidized hyaluronan was determined by quantifying aldehyde groups formed with t-butyl carbazate titration via carbazone formation [13]. A solution of the oxidized hyaluronan (10 mg/ml in pH 5.2 acetate buffer) and a 5-fold excess tertbutyl carbazate in the same buffer were allowed to react for 24 hrs at ambient temperature, followed by the addition of a 5-fold excess of NaBH₃CN. After 12 hrs, the reaction product was precipitated three times with acetone and the final precipitate was dialyzed thoroughly against water, followed by lyophilization. The degree of oxidation (i.e., abundance of aldehyde groups) was assessed using ¹H NMR by integrating the peaks: 1.32 ppm (tert-butyl) and 1.9 ppm (CH₃ of hyaluronic acid).

### Example 6

### Preparation of Acrylated Chitosan

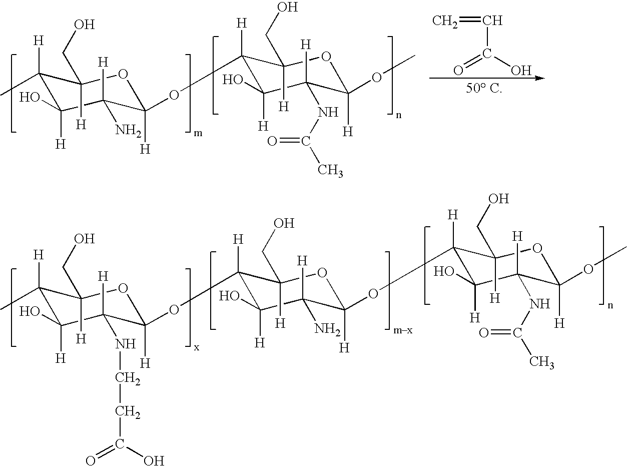

5.52 ml of acrylic acid was dissolved in 150 ml of double distilled water and 3 g of chitosan (Kraeber® 9012-76-4, molecular weight 200-600 kD) was added to it. The mixture was heated to 50° C. and vigorously stirred for 3 days. After removal of insoluble fragments by centrifugation, the product was collected and its pH was adjusted to 11 by adding NaOH solution. The mixture was dialyzed extensively to remove impurities.

### Example 7

### Preparation of PEG-Chitosan

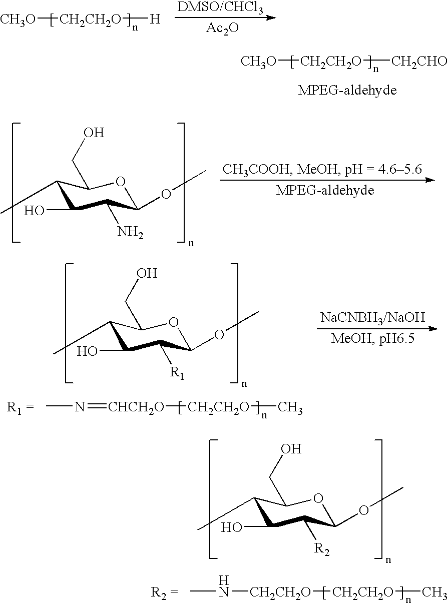

Monomethyl-PEG-aldehyde was prepared by the oxidation of Monomethyl-PEG (MPEG)with DMSO/acetic anhydride: 10 g of the dried MPEG was dissolved in anhydrous DMSO (30 ml) and chloroform (2 ml). Acetic anhydride (5 ml) was introduced into the solution and the mixture is stirred for 9 h at room temperature. The product was precipitated in 500 ml ethyl ether and filtered. Then the product was dissolved in chloroform and re-precipitated in ethyl ether twice and dried.

Chitosan (0.5 g, 3 mmol as monosaccharide residue containing 2.5 mmol amino groups, Kraeber 9012-76-4, molecular weight 200-600 kD) was dissolved in 2% aqueous acetic acid solution (20 ml) and methanol (10 ml). A 15 ml sample of MPEG-aldehyde (8 g, DC: 0.40) in aqueous solution was added into the chitosan solution and stirred for 1 h at room temperature. Then the pH of chitosan/MPEG-monoaldehyde solution was adjusted to 6.0-6.5 with aqueous 1 M NaOH solution and stirred for 2 h at room temperature. NaCNBH₃ (0.476 g, 7.6 mmol) in 7 ml water was added to the reaction mixture dropwise and the solution was stirred for 18 h at room temperature. The mixture was dialyzed with dialysis membrane (COMW 6000-8000) against aqueous 0.5 M NaOH solution and water alternately. When the pH of outer solution reached 7.5, the inner solution was centrifuged at 5,000 rpm for 20 min. The precipitate was removed. The supernatant was freeze-dried and washed with 100 ml acetone to get rid of unreacted MPEG. After vacuum drying, the final product (white powder) was obtained as water soluble or organic solvent soluble PEG-g-Chitosan. The yield of water soluble derivatives was around 90% based on the weight of starting chitosan and PEG-aldehyde.

### Preparation of Formulations of the Invention and Testing on Human Skin:

### Example 8

A solution of PEG-chitosan (2.5 wt %) and hyaluronan (0.5%) in water at pH in the range of about 3.5 to about 5.5 was made up by first making solutions of the two polymers independently at the stated pH, then mixing the solutions. The mixture rapidly formed a viscous solution, which was applied with a syringe to the backs of one hand of each of 13 volunteer test subjects, who gently rubbed the formulation onto the skin. After several minutes, the formulation was removed and the skin area washed with water. All test subjects reported that their subjective impression was that the area of skin to which the formulation had been applied was markedly more smooth and soft than prior to application of the formulation.

### Example 9

A solution of PEG-chitosan (1.3% w/v, 25 g), hyaluronan (2% w/v, 2.3 g), and 0.1 M HCl (pH 1.45, 0.45 g) was made up. The viscosity increased markedly within about 30 seconds. The pH of the resulting mixture was within the 3.5 to 5.5 range. The viscous solution, when applied to skin, resulted in skin that was subjectively evaluated as more smooth and soft after removal of the mixture and water washing.

### Example 10

A solution of PEG-chitosan (1% w/v, 10 g), hyaluronan (1% w/v, 1 g) and 0.1 M HCl (pH 1.5, 0.3 g) was made up. The viscosity increased markedly within about 30 seconds. The pH of the resulting mixture was within the 3.5 to 5.5 range. The viscous solution, when applied to skin, resulted in skin that was subjectively evaluated as more smooth and soft after removal of the mixture and water washing.

While the invention has been described and exemplified in sufficient detail for those skilled in this art to make and use it, various alternatives, modifications, and improvements will be apparent to those skilled in the art without departing from the spirit and scope of the claims.

We claim:

1. A cosmetic formulation for skin care or beautification, the formulation comprising an alkylated chitosan derivative, a polybasic carboxylic acid or an oxidized polysaccharide, and water.

2. The formulation of claim 1 wherein the alkylated chitosan derivative comprises an acrylated chitosan or a poly(oxyalkylene)chitosan.

3. The formulation of claim 1 wherein the polybasic carboxylic acid or the oxidized polysaccharide comprises hyaluronic acid, carboxymethyl cellulose, oxidized dextran, oxidized starch, oxidized hyaluronic acid, or a low molecular weight di-, tri-, or tetra-carboxylic acid.

4. The formulation of claim 3 wherein the low molecular weight di-, tri-, or tetra-carboxylic acid comprises a (C₃-C₁₂)-alkane-α,ω-dicarboxylic acid, citric acid or ethylenediamine tetraacetic acid.

5. The formulation of claim 1 wherein the formulation comprises poly(oxyethylene)chitosan.

6. The formulation of claim 1 wherein the formulation comprises oxidized dextran or oxidized hyaluronic acid.

7. The formulation of claim 1 wherein the polybasic carboxylic acid or oxidized polysaccharide comprises about 0.1 wt % to about 5 wt % of the formulation.

8. The formulation of claim 1 wherein the alkylated chitosan comprises about 0.1 wt % to about 10 wt % of the formulation.

9. The formulation of claim 5, wherein the formulation further comprises hyaluronan, and wherein the pH of the formulation is about 3.5 to about 5.5.

10. A method of moisturizing or softening skin of a human being, comprising holding the formulation of claim 1 in close contact with the skin.

11. The method of claim 10 wherein the formulation is held in close contact with the skin for a period of time of about 10 seconds to about 60 minutes.

12. The method of claim 10, further comprising initially preparing a first solution comprising the alkylated chitosan derivative dissolved in water and a second solution comprising the polybasic carboxylic acid or oxidized polysaccharide dissolved in water and then mixing the first solution and the second solution in situ on the skin or immediately prior to emplacement on the skin.

13. The method of claim 12, wherein the mixture of the first solution and the second solution increases substantially in viscosity immediately subsequent to the step of mixing.

14. The method of claim 10, wherein the alkylated chitosan derivative comprises PEG-chitosan, the polybasic carboxylic acid comprises hyaluronic acid, and the pH of the solution in about 3.5 to about 5.5.

15. A method of preparation of the formulation of claim 1, comprising initially forming a first aqueous solution of the alkylated chitosan and separately a second aqueous solution of the polybasic carboxylic acid or oxidized polysaccharide, then mixing the first and the second aqueous solutions in situ on skin of a human being or immediately prior to application to the skin, to form a mixed solution.

16. The method of claim 15 wherein the formulation has a pH of about 3.5 to about 5.5.

17. The method of claim 15 wherein the mixed solution increases substantially in viscosity within about 12 minutes after mixing.

18. The method of claim 17, wherein the mixed solution increases substantially in viscosity while disposed on the skin.

19. A cosmetic composition comprising the formulation of claim 1 for moisturizing or softening skin.

20. A disposable towel or skin wipe comprising the formulation of claim 1 for moisturizing or softening skin.

21. A facial foam mask comprising the formulation of claim 1 or a dehydrated composition prepared by removing water from the formulation of claim 1, for moisturizing or softening skin.

22. The facial foam mask of claim 21 wherein the step of removing water comprises lyophilization.
